(19) United States
(12) Patent Application Publication
(10) Pub. No.: US 2009/0082693 A1
(43) Pub. Date: Mar. 26, 2009

(54) Method and apparatus for providing temperature sensor module in a data communication system

(75) Inventor: Gary Ashley Stafford, Hayward, CA (US)

Correspondence Address: JACKSON & CO., LLP, 6114 LA SALLE AVENUE, #507, OAKLAND, CA, 94611-2802, US

(73) Assignee: TheraSense, Inc., Alameda, CA (US)

(21) Appl. No.: 11/026,766

(22) Filed: Dec. 29, 2004

(51) Int. Cl. A61B 5/01

(52) U.S. Cl. 600/549

(57) ABSTRACT

Method and apparatus for providing temperature sensor module in a transmitter unit of a continuous glucose monitoring system includes a temperature probe portion provided with thermally conductive material such as thermally conductive epoxy provided in the temperature sensor module that is press fitted, insert molded, heat stacked, or attached by adhesive to the bottom portion of the transmitter housing. The temperature sensor module is substantially in physical contact with the patient's skin, and thus is configured to detect the skin temperature corresponding to each measured blood glucose level from the sensor.

### BACKGROUND

The present invention relates to data detection and monitoring systems. More specifically, the present invention relates to method and apparatus providing skin temperature sensor module in the transmitter unit of data communication systems such as in continuous glucose monitoring systems.

Continuous glucose monitoring systems generally include a sensor such as a subcutaneous analyte sensor for detecting analyte levels such as blood glucose levels, a transmitter (such as an RF transmitter) in communication with the sensor and configured to receive the sensor signals and to transmit them to a corresponding receiver unit by for example, using RF data transmission protocol. The receiver may be operatively coupled to a blood glucose monitor that performs blood glucose related calculations and data analysis.

Typically, the blood glucose data measured by the sensor is effected by the skin temperature in such a way that the accurate skin temperature measurement contemporaneous to the blood glucose measurement is necessary to compensate for the potential temperature related deviation in the blood glucose reading.

In view of the foregoing, it would be desirable to have method and apparatus for skin temperature measurement in a transmitter unit of a data communication system such as continuous glucose monitoring systems. More specifically, it would be desirable to have method and apparatus for skin temperature detection for blood glucose measurement compensation.

### SUMMARY OF THE INVENTION

In view of the foregoing, there is provided a temperature sensor module in the transmitter unit of a continuous glucose monitoring system. More specifically, the transmitter housing is provided with a temperature sensor module mounted thereto on the lower side so as to be in physical contact with the skin. The temperature sensor module may include a temperature probe substantially surrounded by thermally conductive material such as epoxy that are configured to accurately and timely detect the skin temperature of a patient.

In accordance with the various embodiments of the present invention, the temperature sensor module may be press fitted, insert molded, heat stacked or attached by adhesive into the bottom portion of the transmitter housing so as to be in substantial physical contact with the patient's skin. Moreover, the temperature sensor module may include any substantially conductive material such as aluminum, stainless steel, gold, silver, and beryllium copper. The press fitted temperature sensor may be substantially filled with thermally conductive epoxy with a temperature probe provided therein. The temperature probe may be connected by a thermally conductive wire to one or more contact points on the printed circuit board of the transmitter unit. In this manner, the transmitter unit may be configured to determine the temperature of the skin with each blood glucose reading received from the sensor.

### BRIEF DESCRIPTION OF THE DRAWINGS

FIG. 1 illustrates a perspective cross sectional view of a transmitter unit of a continuous glucose measuring system including temperature sensor module in accordance with one embodiment of the present invention;

FIG. 2 is an expanded cross sectional view of the temperature sensor module of the transmitter unit shown in FIG. 1;

FIG. 3 is a side cross sectional view of the transmitter unit housing without the temperature sensor module in accordance with one embodiment of the present invention;

FIG. 4 illustrates a perspective bottom view of the transmitter unit housing shown in FIG. 3;

FIG. 5 illustrates a perspective cross sectional view of the temperature sensor module of the transmitter unit of FIG. 1 in accordance with one embodiment of the present invention; and

FIG. 6 is a perspective view of the temperature sensor module shown in FIG.

### DETAILED DESCRIPTION

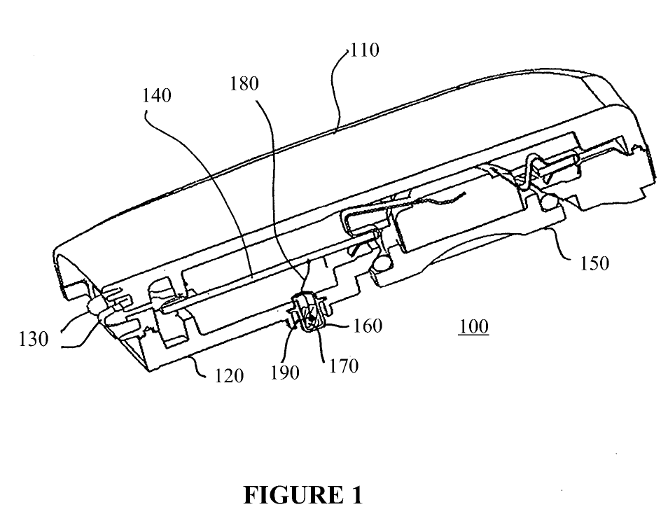

FIG. 1 illustrates a perspective cross sectional view of a transmitter unit of a continuous glucose measuring system including temperature sensor module in accordance with one embodiment of the present invention. Referring to the Figure, the transmitter unit 100 of a continuous glucose monitoring system in accordance with one embodiment of the present invention includes a top housing 110 and a bottom housing 120 correspondingly coupled to the top housing 100. Referring back to the Figure, also provided on the bottom housing 120 of the transmitter is battery cover 150 configured to cover a transmitter battery (not shown) for powering the transmitter unit 100. The battery cover 150 in one embodiment is configured to provide substantially water tight seal so that moisture does not enter the battery compartment covered by the battery cover 150 and into the transmitter unit 100.

Referring back to FIG. 1, the bottom housing 120 of the transmitter unit 100 is further configured to receive a temperature sensor module 160. In one embodiment, when the transmitter is mounted onto the skin of a patient (and operatively coupled to a sensor (such as a subcutaneous sensor) for measuring blood glucose levels of the patient, the temperature sensor module 160 may be configured to substantially and constantly be on physical contact with the patient's skin.

Referring again to FIG. 1, the temperature sensor module 160 in one embodiment includes a substantially hollow space within its outer surface which in one embodiment is filled with thermally conductive material such as thermally conductive epoxy 190 to retain a temperature probe 190 therein. The temperature probed 190 in one embodiment may be further connected to a conductive wire or connection 180. In turn, as shown in the Figure, the conductive wire 180 may be connected to a printed circuit board (PCB) 140 housed within the transmitter unit 100.

Referring yet again to FIG. 1, the transmitter unit 100 in one embodiment is further provided with one or more polymer pins that operate as contact point(s) to a sensor which is configured to detect analyte levels such as blood glucose levels, in a patient.

In this manner, in accordance with one embodiment of the present invention, with the temperature sensor module 160 substantially constantly in physical contact with the skin of the patient on which the transmitter unit 100 is mounted, the temperature sensor module 160 is configured to thermally translate the skin temperature by its inherent thermally conductive characteristics. More specifically, when the skin temperature rises, for example, that rise in temperature is thermally translated by the temperature sensor module 160 which, in turn, translates the rise in temperature to the thermally conductive epoxy 170 within the sensor module 160. Within the scope of the present invention, the shape of the temperature sensor module 160 may a cylindrical barrel shape, a tubular shape, a spherical shape or any other equivalent shape which may substantially retain the thermal characteristics as described herein, and for substantially containing the thermally conducive epoxy 170 (or any other type of thermally conductive material) therein.

Accordingly, the temperature probe 190 placed within the thermally conductive epoxy 170 then detects the change in temperature, and translates that thermal deviation over the conductive wire 180 to the printed circuit board 140 which includes circuitry configured to determine the necessary temperature compensation based on the measured skin temperature corresponding to the detected blood glucose level received from the sensor (not shown).

In the manner described above, in accordance with one embodiment of the present invention, the transmitter unit 100 may be provided with a temperature sensor module 160 to detect skin temperature so as to compensate for the deviation in measured blood glucose levels received from the sensor. Furthermore, in one embodiment, the temperature sensor module 160 is press fitted into the bottom housing 120 of the transmitter unit 100 such that there is provided a substantially water tight seal to retain the water proof characteristics of the transmitter unit 100. Alternatively, the temperature sensor module 160 may be insert molded, heat stacked or attached by adhesive to the bottom housing 120 of the transmitter unit 100.

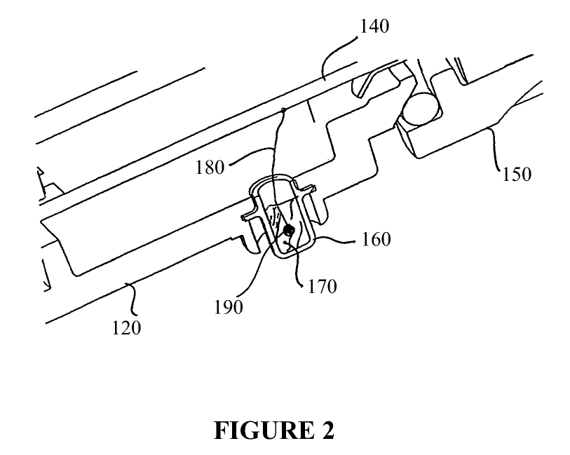

FIG. 2 is an expanded cross sectional view of the temperature sensor module of the transmitter unit shown in FIG. 1. In one aspect of the present invention, the conductive wire 180 connecting the temperature probe 190 to the printed circuit board 140 within the transmitter unit 100 may include flex circuit, zebra strip, or any other functionally equivalent approach to connect the temperature probe 190 within the temperature sensor module 160 to the printed circuit board 140.

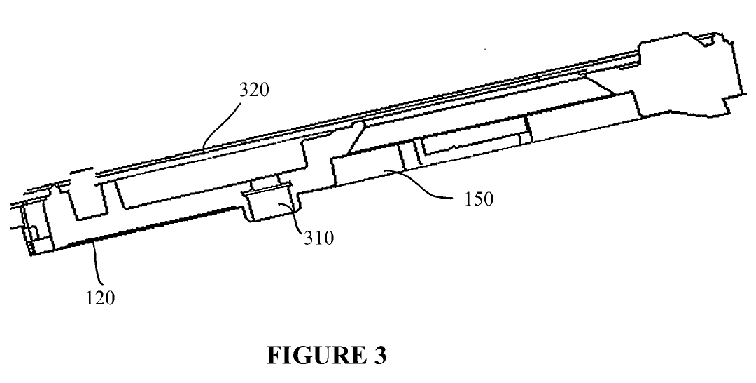

FIG. 3 is a side cross sectional view of the transmitter unit housing without the temperature sensor module in accordance with one embodiment of the present invention. As can be seen in additional detail in FIG. 3, the bottom housing 120 of the transmitter unit 100 is provided with a temperature sensor module receiving section 310 formed thereon, such that the temperature sensor module 310 may be press fitted into the temperature sensor module receiving section 310 to form a substantially water tight seal between the outer surface of the temperature sensor module 160 and the inner surface of the temperature sensor module receiving section 310. Referring back to FIG. 3, also shown is a predefined groove section 320 within the bottom housing 120 configured to receive the printed circuit board 140 (FIG. 1) to substantially rigidly retain the printed circuit board 140 in position within the transmitter unit 100.

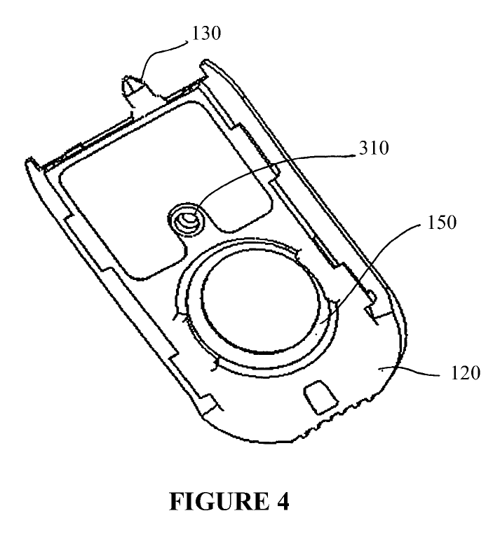

FIG. 4 illustrates a perspective bottom view of the transmitter unit housing shown in FIG. 3. As shown in FIG. 4, it can be seen from the shape of the battery cover 150 that in one embodiment, the transmitter unit 100 may be powered with a disposable battery similar to those used for watches. In one embodiment, since the transmitter unit 100 in the continuous glucose measuring system may be configured to be disposable after each use, the durability of the battery life may not impact the operation of the transmitter unit 100.

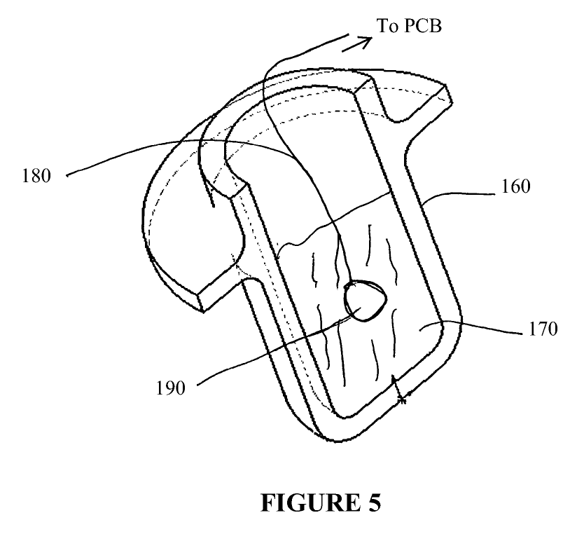

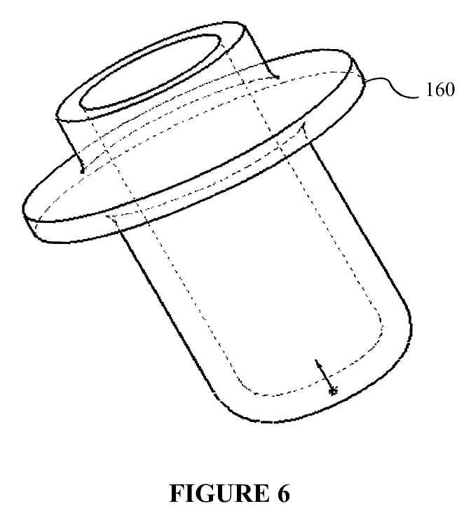

FIG. 5 illustrates a perspective cross sectional view of the temperature sensor module of the transmitter unit of FIG. 1 in accordance with one embodiment of the present invention, and FIG. 6 is a perspective view of the temperature sensor module shown in FIG. 5. Referring to FIGS. 5-6, the thermally conductive epoxy 170 is contained in a substantially cylindrical space within the temperature sensor module 160. However, within the scope of the present invention, the shape of the temperature sensor module 160 as well as the inner hollow shape thereof, may be of any configuration, as long as the desired thermal conductivity is retained, and as long as a substantially water tight seal may be formed once the temperature sensor module 160 is fitted into the bottom housing 120 of the transmitter unit 100.

Indeed, an apparatus including a temperature sensor module includes a housing, a conductive material provided substantially within the housing, and a temperature probe substantially completely disposed within the conductive material in the housing, where a change in temperature of the housing is substantially and contemporaneously translated to the temperature probe.

The housing may include a thermally conductive material including one of stainless steel, aluminum, beryllium copper, gold, silver and plastic, while the conductive material provided substantially within the housing may include conductive epoxy. Also, the temperature probe may include a thermistor.

In a further embodiment, the apparatus may also include a conductive wire having a first end and a second end, the first end of the conductive wire connected to the temperature probe, and the second end of the conductive wire configured to connect to one or more contacts on a circuit board, where the conductive wire may include one of a copper wire and thermocouple connection.

Moreover, the housing may be configured to press fit into a transmitter housing such that a substantially water tight seal is formed. Additionally, the housing may be alternatively configured to be insert molded, heat stacked or attached by adhesive to the transmitter housing to obtain the substantially water tight seal.

An apparatus including a temperature sensor module in accordance with another embodiment of the present invention includes a housing, a thermally conductive epoxy provided substantially within the housing, a temperature probe substantially completely disposed within the thermally conductive epoxy in the housing, and a conductive wire having a first end and a second end, the first end of the conductive wire connected to the temperature probe, and the second end of the conductive wire configured to connect to one or more contacts on a circuit board, where a change in temperature of the housing is substantially and contemporaneously translated to the one or more contacts on the circuit board via the conductive wire.

A method of providing a temperature sensor module may include the steps of providing a conductive material substantially within a housing, and substantially disposing a temperature probe substantially within the conductive material in the housing, where a change in temperature of the housing is substantially and contemporaneously translated to the temperature probe.

In the manner described above, in accordance with various embodiments of the present invention, temperature sensor module 160 in the transmitter unit 100 of a data communication system such as a continuous glucose monitoring system is configured to be in physical contact with the patient's skin. The temperature sensor module 140 may include a temperature probe 190 substantially surrounded by thermally conductive material 170 such as epoxy that are configured to accurately and timely detect the skin temperature of a patient.

As discussed above, in one aspect of the present invention, the temperature sensor module 140 may be press fitted into the bottom housing 120 of the transmitter housing so as to be in substantial physical contact with the patient's skin. The temperature sensor module 140 may include any substantially conductive material so long as the desired thermal conductivity may be achieved. The press fitted temperature sensor module 140 may be substantially filled with thermally conductive epoxy 170 with a temperature probe 190 provided therein. The temperature probe 190 may be thermally connected by a conductive wire, for example 180, extended from the thermally conductive epoxy 190 within the temperature sensor module 140 to one or more contact points on the printed circuit board 140 of the transmitter unit 100. In this manner, the transmitter unit 100 may be configured to determine the temperature of the skin corresponding to each blood glucose reading received from the sensor.

Various other modifications and alterations in the structure and method of operation of this invention will be apparent to those skilled in the art without departing from the scope and spirit of the invention. Although the invention has been described in connection with specific preferred embodiments, it should be understood that the invention as claimed should not be unduly limited to such specific embodiments. It is intended that the following claims define the scope of the present invention and that structures and methods within the scope of these claims and their equivalents be covered thereby.

1. An apparatus, comprising:
an analyte data processing unit including a bottom housing configured for placement on a skin layer of a patient;
a temperature module coupled to the bottom housing, a portion of an outer wall of the temperature module configured to contact the skin layer of the patient, the temperature module including an inner cavity defined by an inner wall;
a conductive material provided substantially within the temperature module such that the inner cavity of the temperature module is filled with the conductive material; and
a temperature probe disposed within the conductive material in the inner cavity of the temperature module, the temperature probe positioned within the inner cavity to not contact the inner wall of the temperature module;
wherein a change in temperature on the skin layer of the patient is substantially and contemporaneously translated to the temperature probe; and further
wherein the analyte data processing unit is configured to selectively modify one or more analyte related signals based on the temperature change.

2. The apparatus of claim 1, wherein the temperature module includes a thermally conductive material including one of stainless steel, aluminum, beryllium copper, gold, silver or plastic or combinations thereof.

3. The apparatus of claim 1 wherein the conductive material includes conductive epoxy.

4. The apparatus of claim 1 wherein the temperature probe includes a thermistor.

5. The apparatus of claim 1 further including a conductive wire having a first end and a second end, the first end of the conductive wire connected to the temperature probe, and the second end of the conductive wire configured to connect to one or more contacts on a circuit board.

6. The apparatus of claim 5 wherein the conductive wire includes one of a copper wire or thermocouple connection.

7. The apparatus of claim 1 wherein the temperature module is configured to one of press fit, insert mold, heat stack, and attach by adhesive, to the bottom surface such that a substantially water tight seal is formed.

8. An apparatus, comprising:
an analyte data processing unit including a bottom housing configured for placement on a skin layer of a patient;
a temperature module coupled to the bottom housing, a portion of an outer wall of the temperature module configured to contact the skin layer of the patient, the temperature module including an inner cavity defined by an inner wall;
a thermally conductive epoxy provided substantially within the temperature module such that the inner cavity of the temperature module is filled with the conductive epoxy;
a temperature probe disposed within the thermally conductive epoxy in the inner cavity of the temperature module, the temperature probe positioned within the inner cavity to not contact the inner wall of the temperature module; and
a conductive wire having a first end and a second end, the first end of the conductive wire connected to the temperature probe, and the second end of the conductive wire configured to connect to one or more contacts on a circuit board disposed in the analyte data processing unit;
wherein a change in temperature on the skin layer of the patient is substantially and contemporaneously translated to the one or more contacts on the circuit board via the conductive wire; and further
wherein the analyte data processing unit is configured to selectively modify one or more analyte related signals based on the temperature change.

9. The apparatus of claim 8, wherein the temperature module includes a thermally conductive material including one of stainless steel, aluminum, beryllium copper, gold, silver or plastic or combinations thereof.

10. The apparatus of claim 8 wherein the temperature probe includes a thermistor.

11. The apparatus of claim 8 wherein the conductive wire includes one of a copper wire or thermocouple connection.

12. The apparatus of claim 8 wherein the temperature module is configured to one of press fit, insert mold, heat stack, or attach by adhesive, to a transmitter unit such that a substantially water tight seal is formed.

13. A method, comprising:
disposing an analyte data processing unit on a skin surface of a patients
providing a conductive material substantially within an inner cavity of a temperature module defined by an inner wall, the temperature module coupled to the analyte data processing unit; and
disposing a temperature probe substantially within the conductive material in the inner cavity of the temperature module such that the temperature probe does not contact the inner call of the temperature module;
wherein a change in temperature of the temperature module is substantially and contemporaneously translated to the temperature probe such that one or more analyte related signals received by the analyte data processing unit is selectively modified based on the temperature change.

14. The method of claim 13, wherein the temperature module includes a thermally conductive material including one of stainless steel, aluminum, beryllium copper, gold, silver or plastic or combinations thereof.

15. The method of claim 13 wherein the conductive material includes conductive epoxy.

16. The method of claim 13 wherein the temperature probe includes a thermistor.

17. The method of claim 13 further including the steps of:
connecting a first end of a conductive wire to the temperature probe; and
configuring a second end of the conductive wire for connection to one or more contacts on a circuit board.

18. The method of claim 17 wherein the conductive wire includes one of a copper wire or thermocouple connection.

19. The method of claim 13 further including the step of coupling the temperature module to the analyte data processing unit such that a substantially water tight seal is formed.

20. The method of claim 13 wherein the step of coupling the temperature module to the analyte data processing unit includes one of press fitting, insert molding, heat stacking, or attaching by adhesive, the temperature module to the analyte data processing unit.
